(19) United States
(12) Patent Application Publication
(10) Pub. No.: US 2021/0038126 A1
(43) Pub. Date: Feb. 11, 2021

(54) MULTIMODAL NEEDLE

(71) Applicant: THE FRANCIS CRICK INSTITUTE LIMITED, London, Greater London (GB)

(72) Inventors: Romeo-Robert RACZ, London, Greater London (GB); Andreas SCHAEFER, London, Greater London (GB)

(21) Appl. No.: 16/978,055

(22) PCT Filed: Mar. 6, 2019
(86) PCT No.: PCT/GB2019/050618

§ 371 Date: Sep. 3, 2020

(30) Foreign Application Priority Data
Mar. 7, 2018 (GB) 1803672.3

(51) Int. Cl. A61B 5/1473; A61B 5/00

(52) U.S. Cl. CPC A61B 5/1473; A61B 2562/028; A61B 5/6848

(57) ABSTRACT

A multimodal needle comprises a plurality of micro-electrodes for electrochemical sensing. Each micro-electrode comprises a core of conducting material, insulating material surrounding the core, and a layer of metal or metal oxide nano-structures deposited on tips of the micro-electrodes at a first end for interfacing with a target sensing site. Such a multimodal needle is particularly useful for identifying tissue around the needle tip.

The present invention relates to the field of medical devices for insertion into human or animal body, in particular needles.

Insertion of needles into human body generally relies on a practitioner's knowledge and experience. Whilst this may be sufficient in some circumstances, the success rate would improve significantly and accidental damage reduced if a needle-tip positioning read-out would be available to the practitioner. For example, whilst attempting to deliver anesthetics by inserting a needle into a tissue and placing the needle tip at the target location, iatrogenic injuries can occur by penetration of a nerve fascicle, which may be further aggravated by any subsequent drug release. Furthermore, in some procedures such as radiofrequency ablation using radiofrequency needles it can be difficult to distinguish while using positron-emission tomography or computer tomography between tissues that needs to be treated and those that have been ablated, which can lead to multiple invasive procedures.

At least some examples provide a multimodal needle comprising a plurality of micro-electrodes for electrochemical sensing, where each micro-electrode comprises: a core of conducting material, insulating material surrounding the core, and a layer of metal or metal oxide nano-structures deposited on tips of the micro-electrodes at a first end for interfacing with a target sensing site.

The micro-electrodes can be used for sensing electrical and chemical events of biological systems, such that it can be used to record bioelectrical events, to determine biologically significant substance or substances (e.g. proteins, neurotransmitters, hydrogen peroxide, calcium, nitric oxide, DNA), label-free affinity impedimetric biosensing (capacitance and resistance measuring) or for electrophysiological applications, tumor scanning and electrotherapy or even cardiovascular scanning, for example.

The metal or metal oxide nano-structures deposited on tips of the micro-electrodes at the first end for interfacing with the target sensing site help reduce impedance at the first end tip of the micro-electrodes.

The term “needle” is used herein to include any medical device for being inserted into human or animal body. For example, the needle may comprise a generally elongated structure with a first end for being inserted into a human or animal body. In some embodiments the first end comprises a sharp or pointed end. In other embodiments, the first end does not comprise a sharp or pointed end, such as a probe. In this invention the needle may be inserted to obtain information about the tissue in contact with the first end of the needle by electrochemical sensing. The multimodal needle of present invention comprises multiple modalities in that, for example, the needle is configured to provide readings of a target sensing site whilst also being able to remove from or inject to the target sensing site. In other examples, the needle may provide radiofrequency targeting as well as providing recordings of a target sensing site. The target sensing site may be located in the needle tip region, such that the target sensing site relates to recordings of regions in proximity of the needle tip.

The micro-electrodes of the multimodal needle may have a diameter at the micron scale. More particularly the micro-electrodes may have a diameter less than or equal to 30 μm; or less than or equal to 25 μm, or less than or equal to 20 μm, or less than or equal to 15 μm, or less than or equal to 10 μm. Given the micron scale of the electrodes, this means that a higher resolution reading may be obtained due to a high channel count compared to current techniques, where only a single electrode in the form of the needle itself may be used.

The multimodal needle may comprise an opening at the tip and a through passage. The through passage may be along the length of the needle, where a first end of the through passage is connected to the opening at the tip and a second end of the through passage is connected to another opening which may be an inlet or an outlet. For example, the main body of the multimodal needle may have structural features of a hypodermic needle or a standard Tuohy epidural needle. In other examples, the needle may comprise the features of a radiofrequency needle.

As the skilled user would appreciate, any other various types of needle structure may be used as necessary, depending on the required use. For example, the needle radius, tip curvature or whether there is an opening and through passage or not may be determined depending on the purpose and/or intended use of the multimodal needle.

The multimodal needle may comprise a functionalization layer deposited on the layer of metal or metal oxide nano-structures at said first end of the micro-electrodes.

The functionalization layer may be a layer for adapting the micro-electrode to a particular electrochemical application. Different functionalization layers may be for different biosensing or electrophysiological purposes. For example, the functionalization layer may be formed of iridium oxide, other metal oxides such as titanium dioxide, manganese oxides, carbon nanotubes, graphene, ATP, DNA, proteins etc., depending on the desired sensing modality of the needle.

Another example of a functionalization layer may comprise self-assembled monolayers. Self-assembly describes the spontaneous formation of discrete nanometric structures from simpler subunits. The simplest self-assembled systems are self-assembled monolayers (SAMs). SAMs are formed by the adsorption of molecules on solid surfaces and are governed by intermolecular forces. By covering the layer of metal or metal oxide nanostructures on the tip of the micro-electrodes with SAMs, the tip may be functionalized for building up a highly specific bio-sensitive layer. This can enable the identification of DNA fragments, biomolecules or analytes present in tissue, bodily fluids, nerves, or serum.

In some examples the needle may be provided without any functionalization layer. In this case, a downstream user of the needle may add the desired functionalization layer themselves depending on the desired sensing modality of the needle.

The tip of the micro-electrode at the first end may comprise a recess, and the layer of metal or metal oxide nano-structures may be deposited on the inside of the recess. The functionalization layer may also be deposited on the inside of said recess.

In some examples, the functionalization material may also protrude out of the recess beyond the end of the electrode tip. Providing a recess in this manner means that a greater volume of functionalization material can be deposited at the end of the electrode, which can improve the electrochemical properties of the needle. Furthermore, the recess provides robustness against mechanical deterioration of the electrode tips.

The micro-electrodes may comprise a connection layer of metal nano-structures deposited on tips of the micro-electrodes at a second end. The second end tips of the micro-electrodes are connectable to an integrated circuit comprising a plurality of contact portions to receive an electrode signal. The connection layers of metal nano-structures on the micro-electrode at the second end may be in contact with corresponding contact portions of the integrated circuit.

A similar recess may be formed at the back end of the micro-electrode, with the connection layer of metal nanostructures formed at least partly inside the recess. Again, this helps provide robustness against mechanical deterioration, which could otherwise wear away the connection layer.

The multimodal needle may comprise a first micro-electrode with a first type of functionalization layer deposited on the layer of metal or metal oxide nano-structures at the first end, and a second micro-electrode with a second type of functionalization layer deposited on the layer of metal or metal oxide nano-structures at the first end. There may be a plurality of such first micro-electrodes and second micro-electrodes, forming different subsets of micro-electrodes. Thus, a single needle may make two or more different types of electrochemical measurements.

The plurality of micro-electrodes may be disposed in a common insulating matrix of the insulating material. For example, the micro-electrodes may be glued to a commercially available, off the shelf needle, which may be cylindrical or tubular. In another example, the micro-electrodes may be formed in a matrix fixable to the inner tube of the needle body. The matrix may be fitted by using connectors such as screw, glue, or any other means for fixing bodies together. The matrix may be compressible in some examples such that the matrix of micro-electrodes may be compressed when being fitted into the needle body and due to the elastic resilience, the matrix is secured to the needle body. In other examples, the matrix that the micro-electrodes are disposed in may be sharpened to be used as a needle itself.

The matrix of micro-electrodes may have a shape of a tube, such that its external surface is fitted to the internal surface of the needle body, where an internal channel is provided along the length of the tube-shaped matrix of micro-electrodes for injection or extraction. In other examples, hollow-core channels may additionally be provided such that fluid may be delivered or removed through these channels.

A first end of the needle may have a sharpened tapered end, having a first end needle tip surface. The first end of the needle is the end which is for being injected into a human or animal body. Thus, a sharpened and/or tapered end may be provided such that the first end of the needle tip may easily pierce an initial barrier, such as the skin.

The first end micro-electrode tips may have a polished angled tip surface with a sharped point. At this sharpened point, the metal or metal oxide nano-structures may be formed on the first end micro-electrode tip surface or in the recess of the first end micro-electrode tip surface.

The tip surface refers to a cross-sectional surface of the needle or micro-electrode body.

The first end needle tip surface may be in plane with the first end micro-electrode tip surface. Thus, the functionalization layer portion is maximally exposed to a target sensing site. Furthermore, a smooth injection is possible when the needle is injected.

The first end tips of the micro-electrodes may be movable in relation to the needle tip. For example, the first end tips of the micro-electrodes may be movable between a retracted position, in which the first end tip of the micro-electrodes are retracted within the needle through passage, and an extended position, in which the first end tips of the micro-electrodes are extended further than the first end needle tip.

In other examples, the micro-electrodes are not just movable between two distinct positions but are movable between a number of different positions. For example, the micro-electrodes may be slide-able in relation to the needle body.

Being moveable in relation to the needle body in this manner, the micro-electrodes may be introduced to a tissue prior to the main needle body tip. As the micro-electrodes are generally structurally not as robust as the needle body, the main needle body tip may puncture through any hard barrier and once within softer tissue, the micro-electrodes may be extended further beyond the tip of the needle. In this manner, a region of the tissue slightly ahead of the tip of the needle may be sensed and determined. As the micro-electrodes are significantly smaller than the main needle body, it is possible for the tissue region ahead of the needle tip to be determined so that the needle may be better directed and maneuvered in the tissue.

In some examples, the micro-electrodes may be provided within the needle through passage. In this case, the chances of any structural damage to the micro-electrodes are minimized.

The micro-electrodes may be provided on the outer surface of the needle tip body. In some examples, micro-electrodes may be provided both within the needle through passage and on the outer surface of the needle body.

The positioning of the micro-electrode tip in relation to the needle body determines the positioning of the sensing site, as sensing is performed at the tips of the micro-electrodes where the metal or metal oxide nano-structures, or in addition the functionalization layer is provided. Accordingly, the first end tips of the micro-electrodes may be provided at locations in relation to the needle tip, where fluid may be extracted or injected or where radiofrequency treatment may be carried out. Thus, the areas of interest in relation to the needle tip may be around an external periphery of the first end needle tip, internal periphery of the first needle tip, or different positions along the length of the needle tip and needle body. Depending on the location of the tips of the micro-electrodes, recordings at different sites around the needle tip and/or needle body surrounding tissue may be obtained using the micro-electrodes.

The needle may comprise a portion for coupling with a radio frequency generator. The portion may be provided at the tip region of the needle. In this manner, when the needle is coupled with a radio frequency generator, may be used to perform ablation.

In some examples, a plurality of electrochemical sensor micro-electrodes connectable to a hypodermic needle may be provided. These micro-electrodes may be fitted onto a needle by a downstream user, to form a multimodal needle as discussed above.

In some examples, a system comprising a multimodal needle as discussed above may be provided with an integrated circuit comprising a plurality of contact portions to receive an electrode signal, and an amplifying portion to amplify the electrode signal received at the contact portions, where the connection layers of metal nano-structures on the tips of the micro-electrode at the second end are in contact with corresponding contact portions of the integrated circuit.

The system may comprise a radio frequency generator for coupling with the multimodal needle.

Further aspects, features and advantages of the present technique will be apparent from the following description of examples, which is to be read in conjunction with the accompanying drawings.

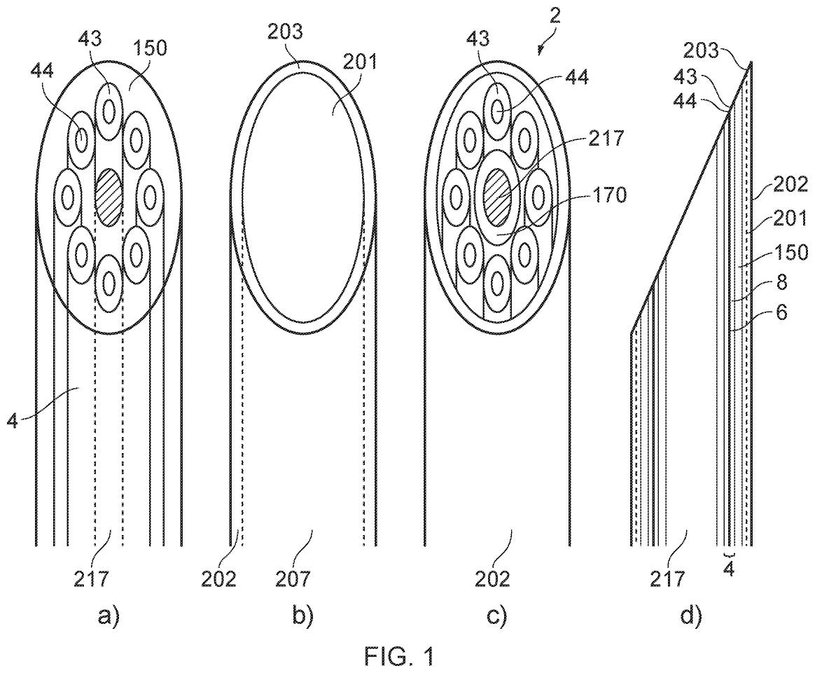

FIG. 1 schematically illustrates an example of a multimodal needle comprising a plurality of micro-electrodes;

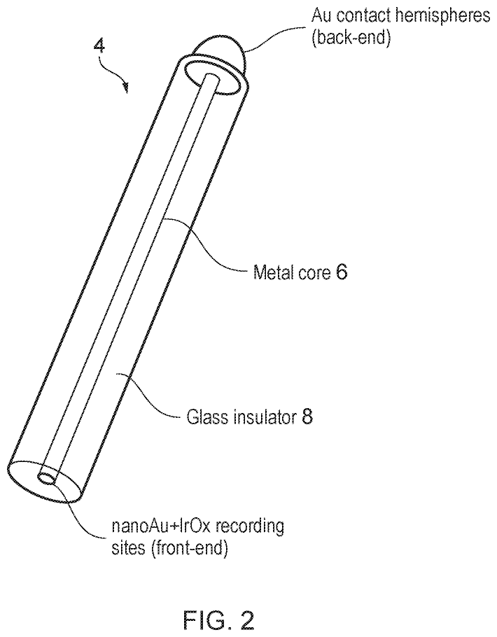

FIG. 2 schematically illustrates a micro-electrode surrounded in insulating material with impedance reducing layer made of gold nano-structures deposited on the tips of the micro-electrode at the first and second ends, and an iridium oxide functionalization layer deposited on the gold nano-structure at the first end of the micro-electrode;

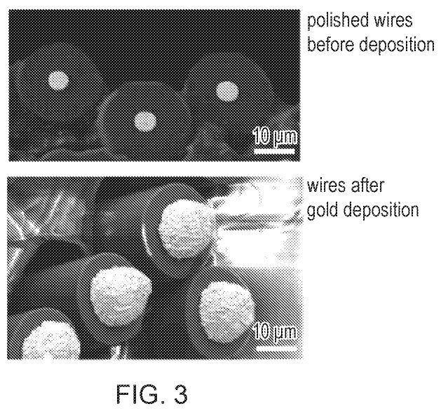

FIG. 3 shows an image of a plurality of micro-electrodes before and after depositing the gold nano-structures;

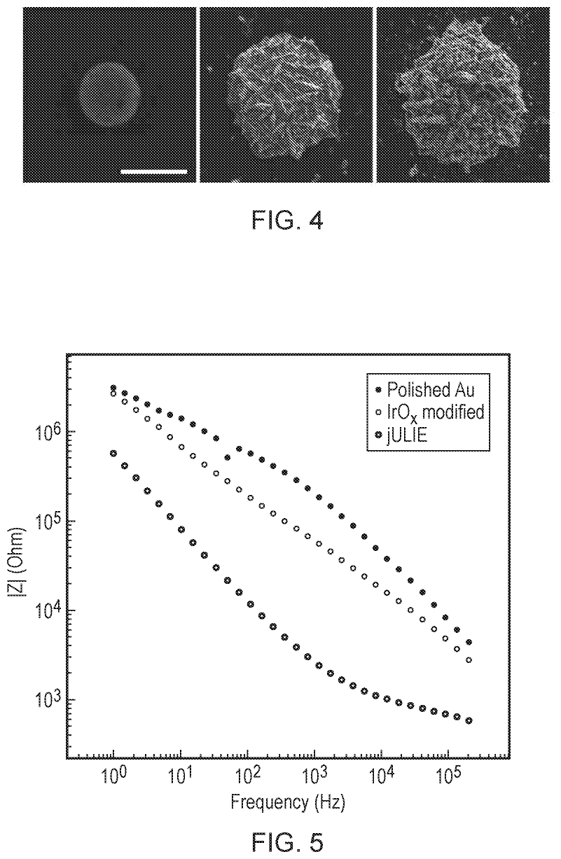

FIG. 4 shows images of the bare metal core wire, the tip of the wire after depositing the gold nano-structures and the tip after depositing the iridium oxide functionalization layer on the gold nano-structures;

FIG. 5 is a graph showing how the impedance at the front end interface of the wires is reduced by including the layer of gold nano-structures;

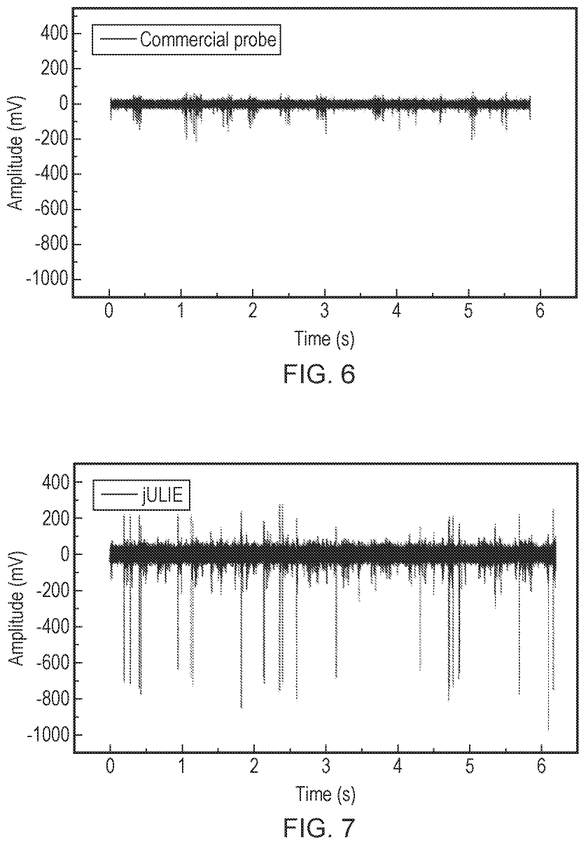

FIGS. 6 and 7 compares signal amplitudes of neuronal recordings measured in a mouse brain using a typical commercial probe and the electrochemical needle of the present technique respectively;

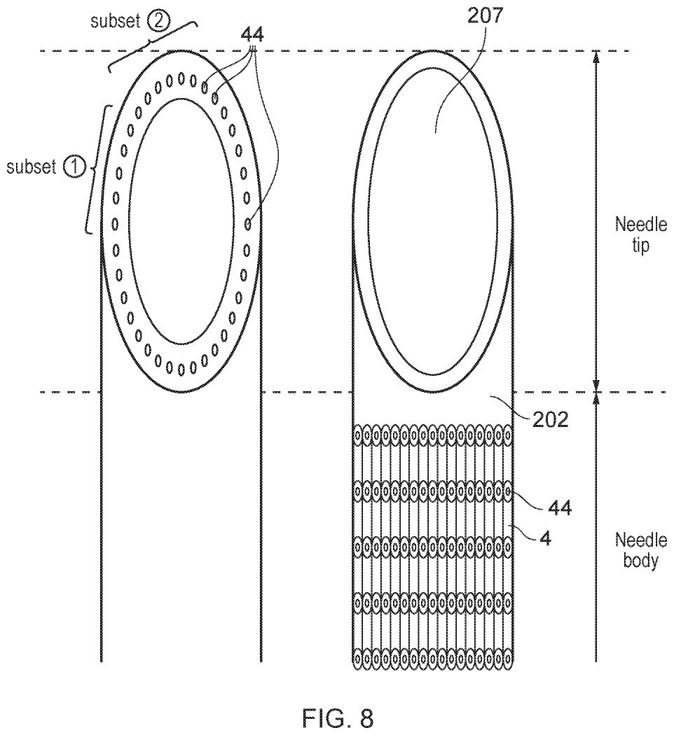

FIG. 8 shows other examples of a multimodal needle comprising a plurality of micro-electrodes;

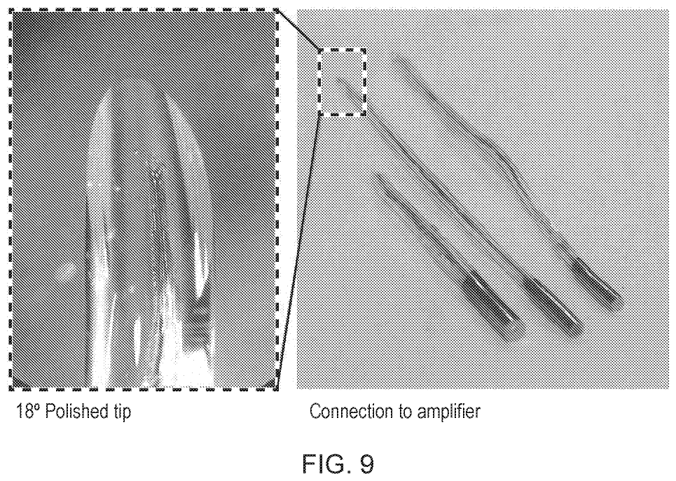

FIG. 9 shows an image of three example stylets of micro-electrodes for being placed in a needle connected to an amplification system on a second end, and another image on the left which is an enlarged image of the tip of the stylet of micro-electrodes;

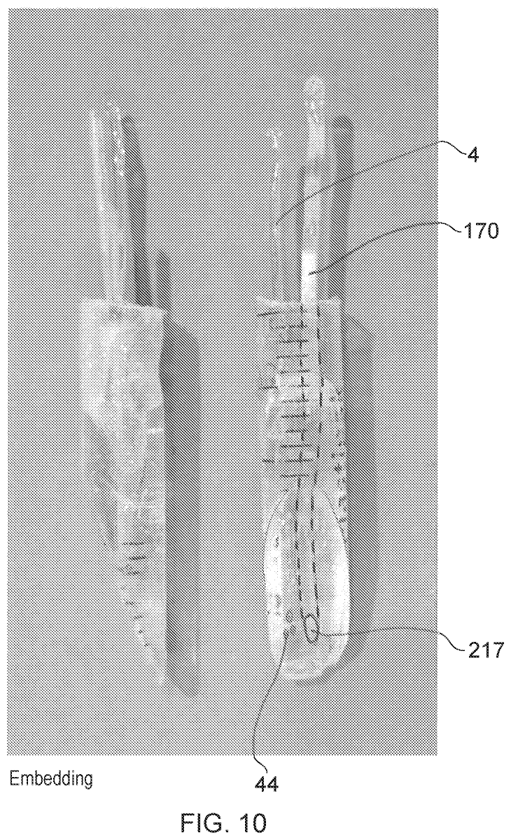

FIG. 10 shows an image of a stylet of micro-electrodes and a channel embedded in an internal through passage of a needle;

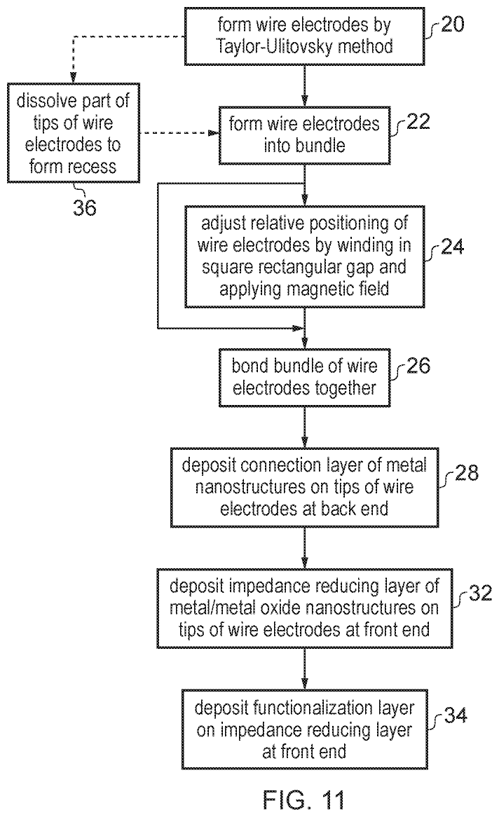

FIG. 11 is a flow diagram illustrating a method of manufacturing an electrochemical needle;

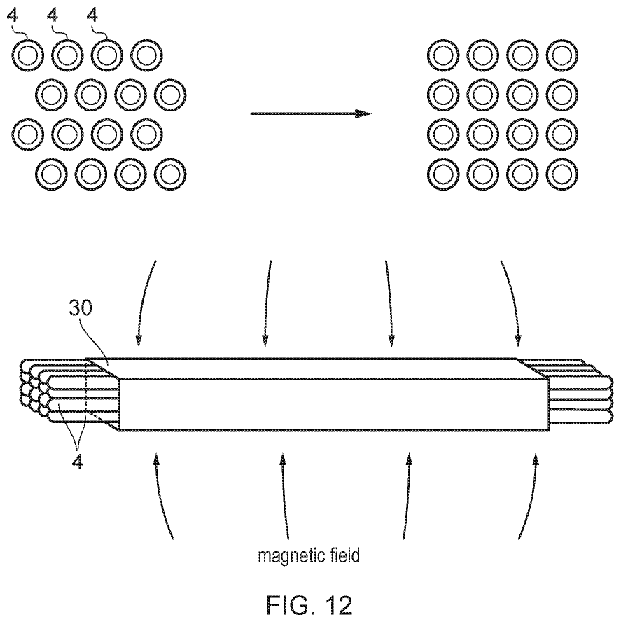

FIG. 12 schematically illustrates an example of adjusting the relative positioning of the wire electrodes in the wire bundle using a magnetic field;

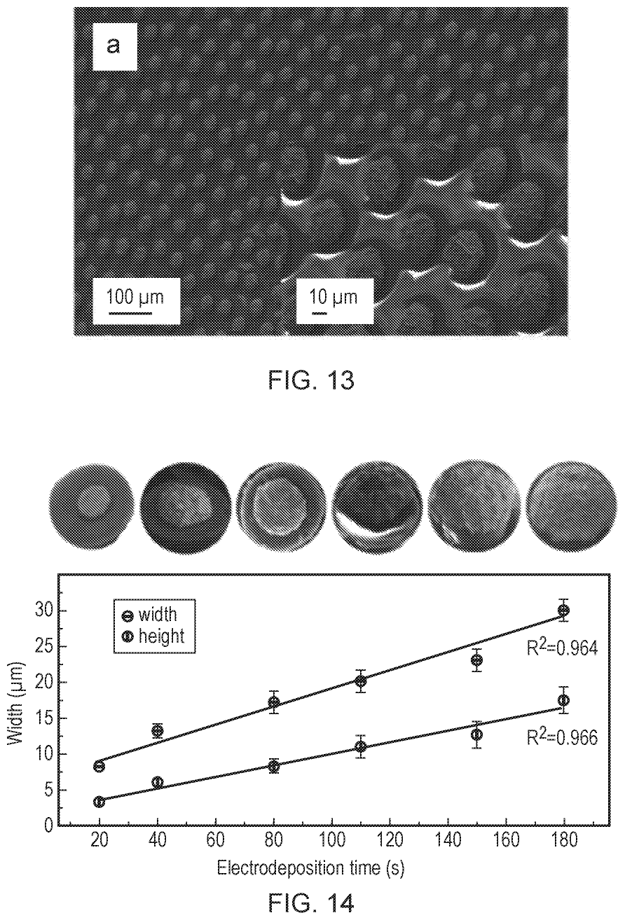

FIG. 13 is an image showing an example where the insulating sheathes of the wire electrodes are melted together to bond the wires together in the bundle;

FIG. 14 is a graph showing variation of the size of gold nano-structure bumps with electrodeposition time;

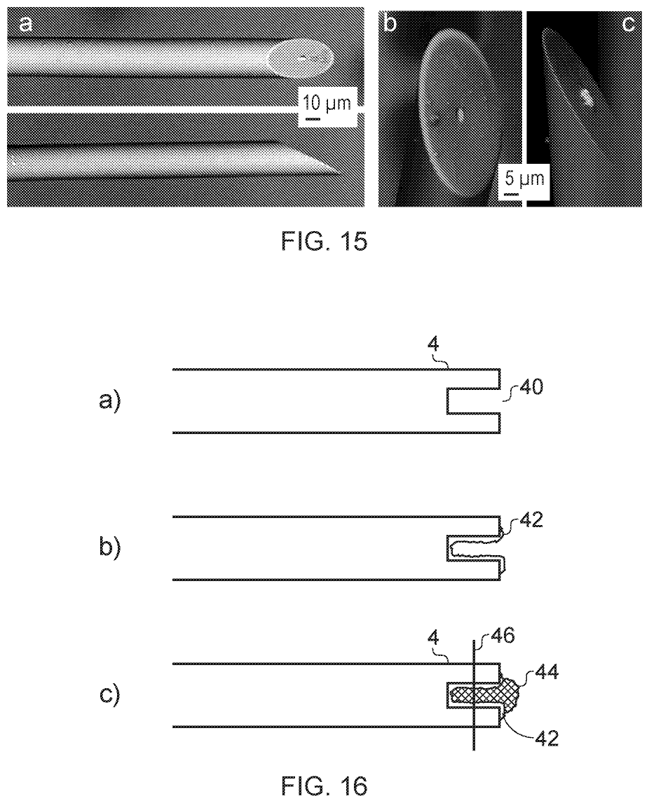

FIG. 15 shows an example of sharpened tips of the wires;

FIG. 16 shows an example where a recess is formed in the tips of the wires and the impedance reducing layer and functionalization layer are deposited on the inside of the recess;

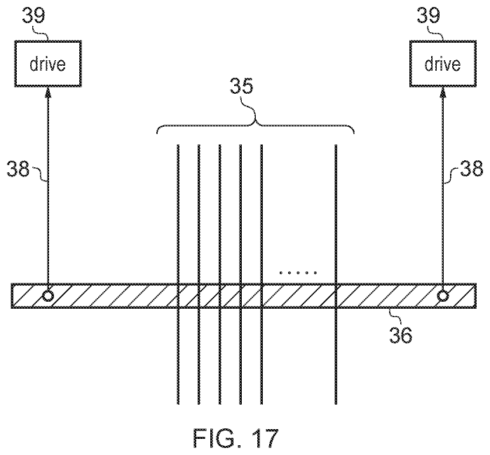

FIG. 17 shows an example in which a harness layer made of piezoelectric material is provided;

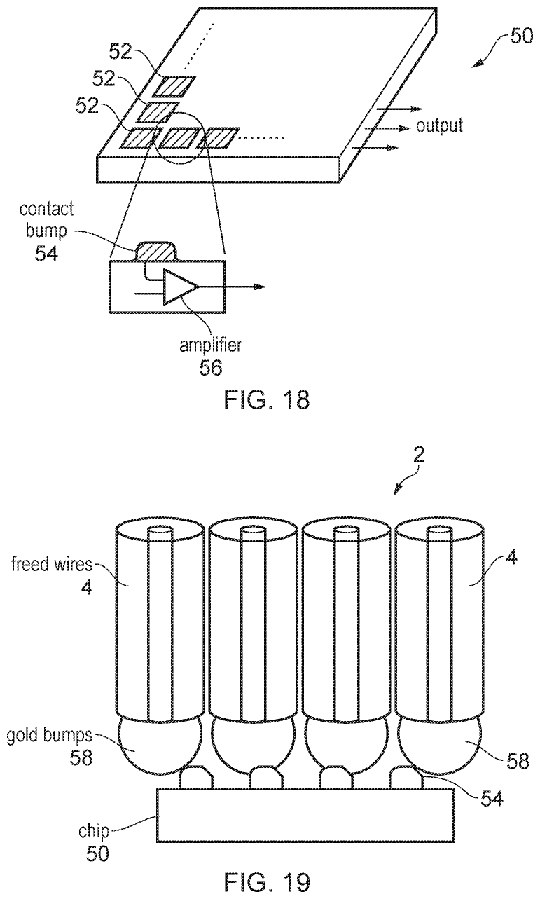

FIG. 18 shows an example of an integrated circuit;

FIG. 19 shows an example of an apparatus in which the integrated circuit is used to read out and amplify the signals received from corresponding wire electrodes of the electrochemical needle;

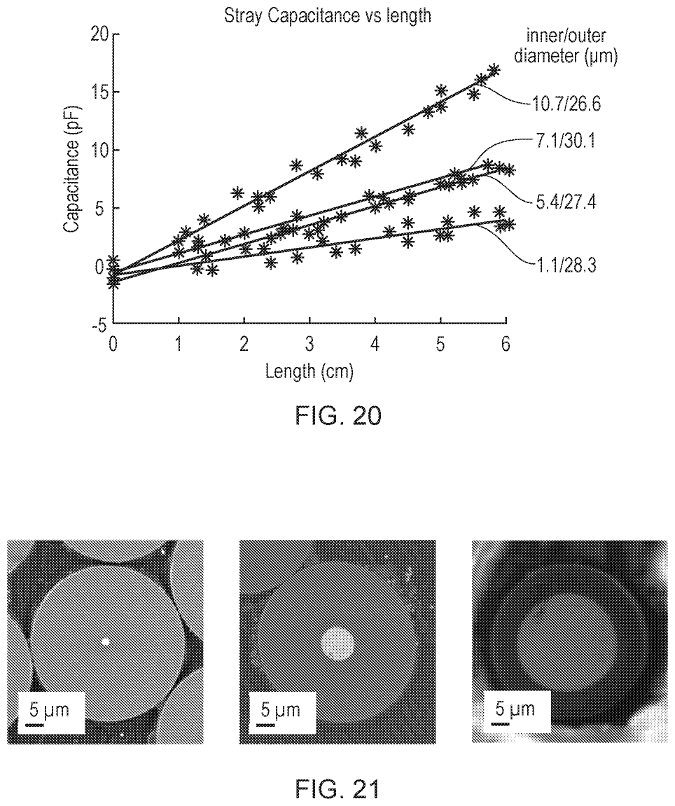

FIG. 20 shows experimental results showing how the stray capacitance at tips of the wire electrodes scales with length of the electrode and with different ratios of core diameter to total diameter;

FIG. 21 shows images of examples with different ratios of core diameter to total diameter;

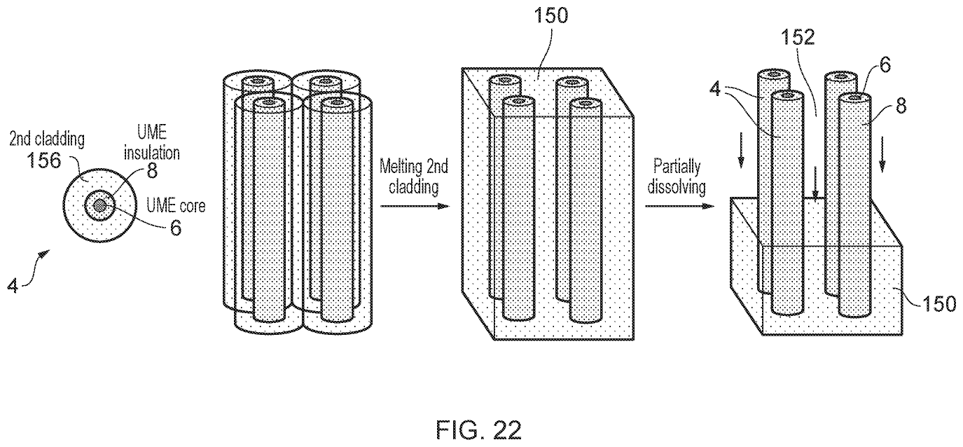

FIG. 22 shows an example of manufacturing a bundle of wires for insertion in the main body of the needle in which the wire electrodes are partially embedded in cladding material along part of the length of the wire electrodes;

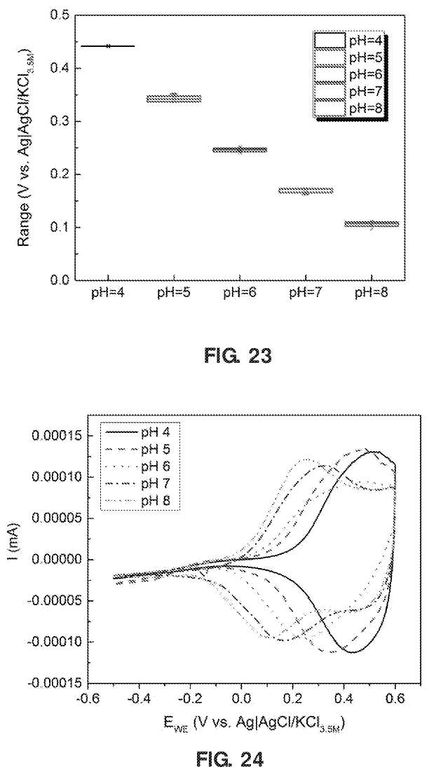

FIG. 23 shows measurements of pH in commercial chemical calibration solutions using the micro-electrodes;

FIG. 24 shows cyclic voltammetry measurements when the micro-electrodes are in chemical calibration solutions of different pH;

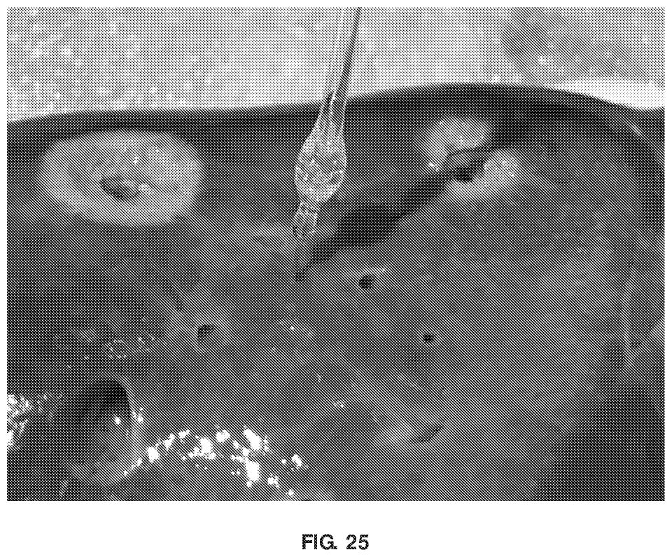

FIG. 25 shows in-vitro validation of a sensor comprising the micro-electrodes for detection of altered tissue during simulated thermal ablation;

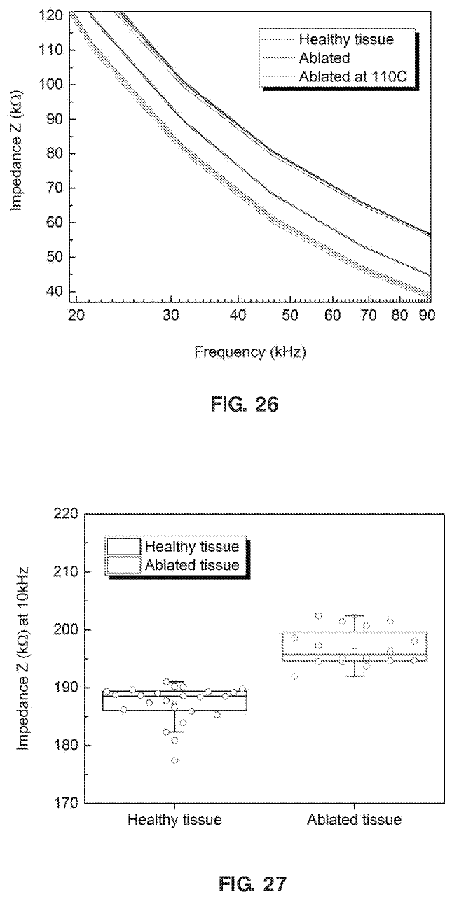

FIG. 26 shows the sensor response to normal and ablated tissue; and

FIG. 27 shows results of measurements to verify the reproducibility of detection of normal or ablated tissue.

FIG. 1 illustrates an example of a multimodal needle 2 comprising a plurality of micro-electrodes 4 for electrochemical sensing. FIG. 1 a) illustrates a plurality of micro-electrodes 4 in a form of a stylet comprising a channel 217 for fluid transfer along the length of the stylet. The stylet is formed by disposing a plurality of micro-electrodes 4 in a matrix 150. The stylet of micro-electrodes may also be referred to as a bundle of micro-electrodes or an electrochemical probe.

Such a stylet of micro-electrodes as shown in FIG. 1 a) can be placed within a through passage 207 of an empty needle as illustrated in FIG. 1 b) to form a multimodal needle 2 of FIG. 1 c). FIG. 1 d) illustrates a cross-sectional view of the multimodal needle 2 of FIG. 1 c).

It is noted that the stylet illustrated in FIG. 1 a) and the stylet inserted in FIG. 1c) are slightly different. For example, whilst the stylet of FIG. 1 a) comprises a channel 217 formed within a matrix 150 that the micro-electrodes are disposed in, the stylet of FIG. 1 c) comprises a hollow-channel tube which is additionally provided and disposed in the matrix 150 parallel to micro-electrodes to provide the channel 217.

As will be appreciated by the skilled reader, the needle may have different shapes of bevels, or the tip may be slightly bent, for example in Tuohy needle or an epidural needle, or the needle tip portion may comprise a radiofrequency receiving portion. The shape of the stylet may vary depending on the shape of the needle and the desired location of the sensing sites 44 on the first end tip of the micro-electrode.

Furthermore, although the micro-electrodes are provided in a form of a stylet which is attachable to the inner surface of a main body of the needle, the micro-electrodes may be provided in other forms. For example, rather than being provided as a bundle of micro-electrodes, each micro-electrodes may be glued to an inner surface of the main needle body or be embedded in the main needle body with the recording sites being exposed, thus above the tip surface 203 of the main needle body. In other examples, the micro-electrodes may be provided in a form of a stylet which is attachable to the outer surface of the main body of the needle. The stylet for outer surface attachment may be provided in a tubular shape so as to tightly fit around the outer surface of the main body.

Describing the multimodal needle 2 as illustrated in FIG. 1 in more detail now, the needle 2 comprises a main body and a bundle of micro-electrodes 4. The bundle of micro-electrodes is provided as a single layer of micro-electrodes in a tubular bundle, where the length of the micro-electrodes are in parallel with the length of the tubular bundle. However, the bundle of micro-electrodes may be formed of different shapes, as will be described in more detail below in relation to FIG. 13. For example, the micro-electrodes 4 may be arranged alongside each other, or the wires could be arranged in a bundle in a regular pattern (such as a square/rectangular lattice or stack arrangement, or a hexagonal packed arrangement) or in an irregular pattern. Furthermore, although only 8 micro-electrodes are illustrated, this is because the figures are only for illustrative purposes only, and a much higher number of micro-electrodes may be used. In this way, local readings in various areas within the multimodal needle may be obtained. For example, different readings may be obtained for the extreme tip (furthest from the second end, or the longest edge) and for the base of the tip of the needle (where the tapering starts), or along any other different positions within the circumference of the needle tip opening.

The multimodal needle 2 of FIG. 1 (in particular c) and d)) comprises an opening at the tip with a through passage 217. A first end of the needle, which is the end for being inserted into a human or animal body, has a sharpened tapered end with a first end needle tip surface 203. The main needle body (illustrated in FIG. 1 b)) comprises an internal surface 201 and an external surface 202, with a main body wall between the internal surface 201 and the external surface. The bundle (or stylet) of micro-electrodes 4 may be provided in the internal through passage 207 of the main needle body. The bundle of micro-electrodes 4 also has a tapered and sharpened first ends. Each micro-electrode 4 has a polished angled tip surface 43 with a sharped point, where a sensing site 43 is provided at a first end of the micro-electrode 4. The micro-electrodes 4 and are placed within the main needle body such that the first end needle tip surface 203 is in plane with the first end micro-electrode tip surface 43. The micro-electrodes 4 may be fixed in place within the main needle body by an adhesion providing material such as a glue or other fastening means, for example a screw or a snap fastener provided at a second end of the micro-electrodes and the main needle body.

FIG. 2 is a schematic illustration of one of the micro-electrodes 4 of the multimodal needle 2. The micro-electrode (also referred to as a wire electrode) includes a core 6 made of a conducting material (e.g. a metal or alloy) surrounded by an insulating material 8. The micro-electrode 4 is an ultramicroelectrode (UME) having a diameter less than or equal to 25 μm. In this example the metal core 6 is made of gold, but other examples of conducting materials which could be used include copper, silver, gold, iron, platinum, lead or other metals, as well as crystalline or amorphous alloy compositions such as brass, bronze, platinum-iridium, lead-silver and magnetic alloys such as FeSiB. In this example, the insulating material 8 is glass, but other examples could use plastics or other insulators.

Each micro-electrode of the needle 2 has a front end (also referred to as a first end), which is the end of the micro-electrode which is for being injected into a human or animal body for interfacing with the sensing target, and a back end (also referred to as a second end), which is the end of the micro-electrode for transmitting the signals measured from the sensing target to the signal read out electronics or data processing equipment. At the front end, the wire electrodes 4 each have an impedance reducing layer of gold nano-structures deposited on the tips of the wire electrodes, and an iridium oxide (IrOx) functionalization layer comprising a layer of iridium oxide nano-structures deposited on top of the gold nano-structures. At the back end, the tips of the wire electrodes have a connection layer for connecting to an electrical connector or the read-out electronics. The connection layer in this example is also made of gold nano-structures, but the back end does not have the additional functionalization layer.

FIG. 3 shows an image of the gold nano-structure hemispheres formed on the back ends of the wires and the back end before hemisphere deposition. Each individual single crystal of the hemisphere may have a unit size in the nanometre scale, e.g. smaller than 300, 100 or 50 nm for example. On the other hand, the overall hemisphere of nano-structures on the back-end may have a width at the micrometre scale, e.g. around 10-20 μm in this example, and on the front end the hemisphere may have a width not exceeding 20% of the wire core's diameter. As can be seen from FIG. 3, the hemisphere may extend over the insulating sheath of the wires as well as the core material on the back-end to facilitate contact with the integrated circuit providing the electronics for reading out signals from the probe. It will be appreciated that the gold-nanostructure layers formed at the front and back ends of the wire electrodes need not be perfectly hemispherical—in general any mound or bump formed on the tip of the wire electrodes may be sufficient.

FIG. 4 shows images illustrating the various layers deposited at the tips of the wire electrodes. The left-hand image of FIG. 4 shows the bare polished metal core prior to depositing either of the layers onto the tip of the wire. The middle image shows the electrode after depositing the gold nano-structure layer. The layer of gold nanostructures has a flaky consistency, providing a large specific surface area for charge transfer which helps to reduce the impedance at the tips of the wire. The right-hand image of FIG. 4 shows an image of the wire electrode after depositing the iridium oxide layer on top of the gold nano-structure layer. The iridium oxide layer has a spongey consistency and provides a surface modification suitable for a range of biosensing or electrochemical applications. For example, the IrOx layer facilitates pH sensing. Also, the chemical properties of iridium oxide provide increased charge storage capacity which enables current injection and amperometric analyte detection (detection of ions in a solution based on electrical current), e.g. for detecting dopamine. 4

For glass ensheathed ultramicroelectrodes (UMEs) to be used in any electrophysiological application which involves reception and transmission of electrical signal through any length, the following characteristics are advantageous:
a controllable frequency response input representing one side usually the one in contact with the biological/and or liquid sample,
a well-insulated and electrical conductive length/body and a low-ohmic connection on the other side, usually the connection side or back-end.
UME connection to any microscopic and/or macroscopic conductor by mechanical means either reversibly or non-reversibly preferably features a repeatedly deformable positively protruding mass from surface.

UMEs feature small stray capacitances (e.g. less than 0.5 pFcm⁻²) given the high insulator-conductor ratio, mechanical workability, broad material choice and commercial availability. UMEs usually have one dimension in the micrometer or nanometer domain and at least one the millimeter or centimeter region, thus the properties of the electrified interfaces are to be carefully considered when high frequency electrical signal need to be passed by micron-sized or nano-sized interfaces.

As mentioned above, it can be of interest to consider local readings at different locations within the needle 2. Providing micro-electrodes such that the first end tips comprising sensing sites are provided at various locations enable this. As will be appreciated, using smaller micro-electrodes in higher numbers enable higher resolution of local readings. However, there are a number of different challenges when attempting to obtain signals using such small micro-electrodes. For example in signal coupling between interfaces, on different conductor lengths towards the resistive junction to finally be delivered and processed by the read out circuitry. The smaller the sensors are, the higher impedances (Z) in aqueous electrolytes become, resulting in significantly weakened signals and high noise levels. The interface's electrical coupling properties consequently bring limitations in the design of the read-out systems. Firstly, more amplifier stages and higher amplifier gains are required to condition recorded signals. Secondly, pre-filter and impedance matching circuitry are included to reduce ambient noise and pick up small-signals. Thirdly, increase in power consumption due to these additional amplifier stages.

These issues can be addressed using the bundle of micro-electrodes of the multimodal needle discussed above. By performing a two-step surface modification of the tips of the UMEs at the front end, to include both a highly fractalized flake-like gold nano-structure layer and a second layer of highly porous metal oxide (e.g. iridium oxide), the impedance at the front end of the electrodes can be greatly reduced. This is shown in the graph of FIG. 5 which compares the impedance across different frequencies for three probes (also referred to as bundle of micro-electrodes):
“polished Au”—a probe made of bare gold metal wires without surface modification of the tips at the front end
“IrOx modified”—a probe where the tips of the wires at the front end have the IrOx layer but not the intervening gold nano-structure layer
“jULIE”—a probe as in the example of FIG. 1 with both the gold nano-structure layer and the IrOx layer at the front end tips of the wires.
   As shown in FIG. 5, the impedance at the front end of the jULIE wires is an order of magnitude lower than the other types of wires. To test jULIEs we performed recordings in the olfactory bulb (OB) of anaesthetized mice (4-6 weeks old, Ketamine/Xylazine anaesthesia) using a Tucker Davis RZ2 amplifier with a PZ2 pre-amplifier and RA16AC-Z headstage. Extracellular spikes were reliably recorded with amplitudes of up to 1.6 mV. Consistent with this, when jULIEs were lowered several mm into the brain and returned to a superficial recording position, extracellular units were reliably recorded throughout the olfactory bulb. Due to the small size of the recording site and minimal damage to the tissue, jULIEs were found to be exceptionally suited for recording large amplitude (500-1500 μV), well isolated signals from the close vicinity of neurons (20-30 μm). FIGS. 6 and 7 show amplitudes of neuronal recordings made in a mouse brain using a typical commercially available probe and the jULIE probe respectively. As is clear from FIG. 7, the amplitudes recorded using the jULIE probe are much larger than the amplitudes shown in FIG. 6 for the commercial probe. Hence, signal to noise ratio can be improved and there is less need for additional amplification.

Other advantages of the probes include:

(i) the dimensions of the penetrating wires are 2× to 5× times smaller and recording sites can be up to 50 times smaller (e.g. 1 μm) than in conventional probes. Also, the use of the Taylor-Ulitovsky method as discussed below results in wires with smoother sides than in conventional probes. This results in reduced tissue displacement and damage as well as in highly localized recordings with better unit separation (better identification of signals from each different locations around the needle).
(ii) the nanostructured interface represents an excellent platform for further improved electrical coupling characteristics with the extracellular media, for example the nanosized gold/IrOx interface allows for substantially higher signal-to-noise with amplitudes of up to 1.5 mV compared to typically 200-500 μV with conventional electrodes.
(iii) the material choice enables semi-automatic preparation for recording sites pre-arrangement to fit anatomical structures; and needle-like sharpening for seamless penetration of the neural tissue.
(iv) there are also substantially improved charge transfer capabilities i.e. enabling current injection for stimulation purposes and neurotransmitters or other analyte monitoring (e.g. alcohol, paracetamol), in a highly localized manner.

FIG. 8 illustrates other examples of a multimodal needle comprising micro-electrodes for electrochemical sensing.

The left hand side illustrates an example multimodal needle configured to have multiple sensing modes. More specifically, the multimodal needle in this example comprises different functionalization layers provided on the micro-electrodes. Differently shaded sensing sites represent use of different functionalization layers. Various types of functionalization layer could be selected according to the desired type of electrochemical measurement.

In general, the functionalization layer may be any layer for adapting the probe to a particular electrochemical application, and may be made from a range of materials. One advantage of using gold as the nano-structure impedance reducing layer is that gold nano-structures provide a good platform for a range of different functionalization layers for different biosensing or electrophysiological purposes. For example, the functionalization layer may include other metal oxides such as titanium dioxide, manganese oxides, carbon nanotubes, graphene, ATP, DNA, proteins etc.

Another example of a functionalization layer may comprise self-assembled monolayers. Self-assembly describes the spontaneous formation of discrete nanometric structures from simpler subunits. During the process of self-assembly, atoms, molecules or biological structures form a more complex secondary layer with fewer degrees of freedom due to packing and stacking. The simplest self-assembled systems are self-assembled monolayers (SAMs). SAMs are formed by the adsorption of molecules on solid surfaces and are governed by intermolecular forces. The most popular molecules forming SAMs are thiols and dithiols: in biology and medicine these molecules are used as building blocks for the design of biomolecule carriers, for bio-recognition assays, as coatings for implants, and as surface agents for changing cell and bacterial adhesion to surfaces. Hence, by covering the layer of metal or metal oxide nanostructures (e.g. the nano-rough gold deposits) on the tip of the microwires with SAMs, we can functionalize the tip and build up a highly specific bio-sensitive layer. This can enable the identification of DNA and RNA fragments, biomolecules or analytes present in tissue, bodily fluids, nerves, or serum.

As can be seen in the left hand side example of FIG. 8, a plurality of different subsets of micro-electrodes having different type of functionalization layer deposited on the top of the metal nano-structure impedance reducing layer at the first end (thus creating the sensing site 44). For example, one subset may be provided with iridium oxide functionalization layer may sense pH or electrical currents, another subset may have DNA probes attached for DNA sensing, and a further subset may have a layer modified with alcohol oxidase for alcohol detection. Different subsets of micro-electrodes may be bonded through an adhesive or a filler layer, or by melting the glass insulating sheaths of the micro-electrodes together. In another alternative, instead of forming the respective subsets separately and then assembling them together, a single micro-electrode bundle could be made with different functionalization layers on respective micro-electrode of the bundle, for example by masking some micro-electrodes during the step of depositing the functionalization layer or by ensuring that the electrodeposition current is only applied to some of the micro-electrodes, with multiple functionalization deposition steps for the different types of functionalization layer.

It should be noted that it is also possible to provide a multimodal needle which does not comprise any functionalization layer. This can then provide a platform on which a downstream user of the probe can add the desired functionalization layer themselves. This approach can be useful for supporting other surface functional modifications using materials which may degrade over time and so need to be applied shortly before their use (e.g. different DNA or RNA probes may be provided on the gold nanostructure impedance reducing layer, for DNA sensing).

The right hand side of FIG. 8 illustrates an example multimodal needle in which the micro-electrodes 4 are provided on the outer surface 202 of the main needle tube body surrounding the needle through passage 207. As can be seen, the first end tips of the micro-electrodes comprising the sensing site 44 are provided at different positions on the outer surface 202. Particularly, the sensing sites 44 are provided along different lengths of the needle body, wherein this may correspond to insertion depth of the needle. Thus, readings may be obtained for different insertion depths. Although the illustration in FIG. 8 shows that the micro-electrodes are provided around the needle body, it is of course possible for the micro-electrodes to be provided around the needle tip portion, where the needle starts to taper and sharpen. It may be particularly useful to provide a recess at the tip of the electrode at the first end for the layer of metal oxide nano-structure and in some cases also the functionalization layer in this recess, to minimise structural damage even when the micro-electrodes are provided on the outer surface of the needle.

FIG. 9 illustrates a micro-electrode stylet connected to an amplification system, and an enlarged image of the tip portion of the micro-electrode stylet. This stylet, if sharpened and treated, could be used as a stand-alone needle. In most cases, however, the stylet is embedded in a needle tip as illustrated in FIG. 10 to form a multimodal needle. In the multimodal needle of FIG. 10, a hollow-channel 217 is provided at the needle tip surface, as the needle tip does not have an opening of its own.

In some examples, the micro-electrodes are moveable in relation to the main needle body, as discussed in relation to FIG. 17 below.

FIG. 11 is a flow diagram illustrating a method of manufacturing an electrochemical probe. At step 20 wire electrodes (also referred to as micro-electrodes) are formed using the Taylor-Ulitovsky method. The Taylor-Ulitovsky method is a technique for forming glass-sheathed wire electrodes with a very fine diameter, e.g. as small as a few microns. In the process, the metal or alloy conducting material is placed inside a glass tube which is closed at one end and the other end of the tube is heated to soften the glass to a temperature at which the conductor melts. The glass can then be drawn down to produce a fine glass capillary with the metal core inside the glass. Hence, metal cores of diameters in the range 1 to 120 microns can be coated with a glass sheath a few microns thick with this method. In particular, wires with a core in the range 1-10 μm surrounded in 10-40 μm of glass can be useful for electrical and electrochemical sensing. The metal used can include copper, silver, gold, iron, platinum, lead or other metals as well as crystalline or amorphous alloy compositions such as brass, bronze, platinum-iridium, or magnetic alloys.

At step 22, the electrodes are formed into a bundle or stack with the wires running parallel to each other. For example, the microwires can be machine wrapped into bundles of 10s, 100s, 1000s, 10000s or 100000s of wire electrodes, to provide multiple channels for recording or cover the available contact portions on an integrated circuit.

At step 24, the relative positioning of the wire electrodes in the bundle is adjusted using a magnetic field, as shown schematically in FIG. 12. As shown in the top part of FIG. 12, when the wire electrodes 4 with a substantially round cross-section are bundled together they will tend to pack together in a hexagonal packed arrangement, in which the electrodes in one row are offset relative to the electrodes in another row. However, as will be discussed below, for reading out the signals measured using the electrodes, it can be useful to interface the wire electrodes with respective contact portions of an integrated circuit (IC), such as those used in multi-electrode arrays (MEA) and other pixelated integrated circuits. Most commercially available ICs have the pixelated contact portions of the readout circuits arranged in a two-dimensional square or rectangular grid pattern. Therefore, to match the industry standard rectangular contact point arrangement of an integrated circuit, it can be useful to reposition the wires in the bundle to form a square or rectangular grid pattern, in which the electrodes form rows and columns as shown in the right hand diagram at the top of FIG. 12. For example, at least, or only, the second end portion of the bundle of micro-electrodes may be provided in such a square or rectangular grid pattern. The lower part of FIG. 12 shows one technique by which this can be done. The wire electrodes 4 are wound through a passageway 30 having a square or rectangular cross section. For example, the passageway 30 may be a tube which could be enclosed on all four sides of the bundle, or could be missing one of the sides (e.g. winding the wires through the inside of a U-shaped bar can be enough). By applying a strong magnetic field (stronger than the electrostatics acting on the wires) to the wires as they pass through the gap, the relative positioning of the wire electrodes can be adjusted to form a square or rectangular grid array. For example, if the wires have gold cores, gold is a diamagnetic material and so a strong enough magnetic field can slightly repel the gold wires, and by pushing them up against the inside of a square or rectangular tube, this forces the wires into the desired square or rectangular grid arrangement. Using a differently shaped tube (for example circular), wires can be forced into a circular grid arrangement.

Alternatively, step 24 can be omitted if the pitch of the wires within the bundle or the size of the gold contacts bumps at the back end of the wires will be sufficient that they can interface with a readout circuit regardless of the hexagonal packed arrangement.

At step 26 of FIG. 11, the bundle of wire electrodes is bonded together. This can be done in various ways. In one example, a filler material or adhesive may be introduced between the respective insulating sheets of the wire electrodes 4 to bond the electrodes together in the bundle. Alternatively, as shown in the example of FIG. 13, the wire electrodes can be bonded together by melting the insulating sheath of respective wires together so as to coalesce the insulator into a common matrix of insulating material surrounding the conducting wire electrodes. For example, this approach can be particularly useful when the insulating material is glass. Hence, as shown in the example of FIG. 13, the individual sheaths of the different wires are no longer visible and instead the wire electrodes are surrounded by a common insulating matrix of glass. This approach can be particularly useful for increasing channel density, because by avoiding the need to include a filler or adhesive between the respective wires, a greater number of electrodes per unit area can be included in the bundle.

Note that the wire electrodes do not need to be bonded along their full length. For example, it can be useful to leave a portion of the wire electrodes nearest the front end of the probe unbonded so that the free ends of the wire electrodes can spread out when connected to or embedded in the main needle body, in accordance with the desired locations of recordings around the needle.

At step 28, a connection layer comprising metal nano-structures is deposited on the tips of the wire electrodes at the back end of the probe. The connection layer can be deposited by electrodeposition, in which the bundle of electrodes is held in a bath of electrolyte and a voltage difference is applied between the wire bundle and another electrode to cause ions in the electrolyte to be attracted to the wire electrode bundle, depositing a coating of metal nanostructures on the tip of each wire.

In one particular example, gold micro-hemispheres were deposited from a two-part aqueous cyanide bath containing 50 gL⁻¹ potassium dicyanoaureate(I) (K₂[Au(CN)₂]) and 500 gL⁻¹ KH₂PO₄ dissolved sequentially in deionized water (18 MOhm) (Tech, UK) at 60° C. All reagents were supplied by Sigma-Aldrich, UK, and were used without further purification. Prior to electrodeposition the polished substrate was washed with ethanol (90%), rinsed with deionized water, wiped with a lint-free cloth (Kimwipes, Kimtech, UK) and dried at 50° C. for 1 hour in an autoclave. The electrodeposition protocol was carried out with a VSP 300 potentiostat-galvanostat (Bio-Logic, France) controlled with EC-Lab (Bio-Logic, France) in a three-electrode cell setup composed of a gold UME bundle as working electrode (W_{E}), a coiled platinum wire (99.99%, GoodFellow, US) as counter electrode (C_{E}) and a Ag/AgCl|KCl/_{3.5M} reference electrode (REF) supplied by BASi, USA (E vs. NHE=0.205V). The REF was kept separated from the bath by a glass tube containing the support electrolyte and a porous Vycor glass separator. During gold deposition the W_{E} potential was kept at E_{red}=−1.1V vs. REF for a time determined according to the desired size of the gold hemisphere to be formed. During electrodeposition the bath was thermostated at 60° C. under vigorous (500 rpm) stirring. This technique has been successful for many different types of metal conductor material, including gold, platinum, tin, copper, brass, bronze, silver and lead.

FIG. 14 is a graph showing how varying the time for which the electrodeposition is performed affects the size of the gold nano-structure bumps formed on the end of the wires. The upper line in FIG. 14 shows variation of the width of the gold bumps with electrodeposition time, and the lower line shows variation of the height of the bumps with electrodeposition time. Hence, the size of the gold bumps can be carefully controlled by varying the electro-deposition time.

Gold can be a particularly useful material for the back end connection layer. In contrast with their applications for the front end sensing, the connection of individual or high-count bundled UMEs to integrated circuitry is poorly examined and represents a significant drawback towards their usability in biomedical applications. Literature offers little or no documentation regarding reversible interfacing methods of individual or UMEs to macroscopic conductors or integrated circuitry, the main practices being based on soldering, conductive silver-epoxy bonding or mercury-dip. Although applied, these methods can easily increase the RC cell time constant at high frequencies given the stray capacitance at the glass-mercury/conductive epoxy junctions and are not relevant for reversible contacting individual or bundled UME assemblies; scaling such practices to high-count UME bundles (up to 1 million, for example) are a considerable engineering challenge. The state-of-the-art indium bump bonding developed for pixelated sensor and read-out chip interconnection employing photolithography, sputtering and evaporation or later electrodeposition could be a suitable processing practice, however due to indium's tensile and ductile properties, mechanical properties and overall tribological behaviour it cannot be applied as a reversible interconnection material in UME interfacing. Copper bumps as interconnects could be considered from a mechanical point of view, however given their possible diffusion into SiO in the presence of an electric field, breaking down transistor reliability, and affinity towards oxidation, make Cu a less attractive candidate as an interconnect material in physiological environments. In contrast, gold is a promising contact material in medium wear conditions which can seamlessly enable reversible, scalable, low-cost, ultra-fine pitch and high yield bumping for interconnection purposes.

At step 32 of FIG. 11, an impedance reducing layer of metal or metal oxide nano-structures is deposited on the tips of the wire electrodes at the front end. This can be done by the same electrodeposition protocol as described above for step 28 for the back end. The material used for the nano-structures at the front end can be the same or different to the material used for the nano-structures at the back end, but in one example both use gold nano-structures.

At step 34 a functionalization layer is deposited on the impedance reducing layer at the front end. Again, this can be deposited by electrodeposition (although other techniques such as spraying could also be used). For example, a layer of metal oxide (e.g. iridium oxide) can be deposited on top of the gold nano-structures at the front end.

In one particular example, the electrodeposition protocol was carried out from a modified electrolyte solution based on a formulation reported by Meyer et al. (2001, “Electrodeposited iridium oxide for neural stimulation and recording electrodes”, Neural Systems and Rehabilitation Engineering, IEEE Transactions on, 9(1), pp. 2-11.), containing 10 gL⁻¹ iridium (IV) chloride hydrate (99.9%, trace metal basis, Sigma-Aldrich, Germany), 25.3 gL⁻¹ oxalic acid dihydrate (reagent grade, Sigma-Aldrich, Germany), and 13.32 gL⁻¹ potassium carbonate (99.0%, BioXtra, Sigma-Aldrich, Germany). Reagents were added sequentially to 50% of the solvent's volume first by dissolving IrCl in the presence of oxalic acid followed by the addition of K₂CO₃ over a 16 hour period until a pH=12 was reached. The electrolyte was aged for approximately 20 days at room temperature in normal light conditions until the solution reached a dark blue colour. IrOx was electrodeposited using a multichannel VSP 300 (Bio-Logic, France) potentiostat-galvanostat in 3 electrode cell setup comprising a glass-ensheathed Au wire bundle as working electrode (WE), a platinum rod (0.5 mm diameter, 99.95%, Goodfellow, US) as counter electrode, and Ag|AgCl|KCl/_{3.5M} (Bioanalytical Systems, US) as a reference electrode (REF). The electrochemical protocol was composed of three consecutive stages combining galvanostatic polarisation (GP), cyclic voltammetry (CV) and pulsed potentiostatic protocols (PP). Between protocols open circuit voltage (OCV) of the WE was monitored for 180 second and represents the steady-state period. During galvanostatic deposition the WE potential was set to 0.8V vs. REF for 500 seconds. During CV deposition the WE potential was swept from −0.5V to 0.60 V vs. REF at 100 mVs in both anodic and cathodic direction. During the pulsed potentiostatic deposition the WE potential was stepped from 0V to 0.60V vs. REF with 1 seconds steps for 500 seconds.

As shown in FIG. 15, the tips of the wire electrodes at the front end can be sharpened to provide a tapered surface that is angled to a point, to facilitate insertion into the brain or other sample material. Different electrodes of the bundle may have the angled surface in different orientations so that when the bundle is inserted into the sample, the angled surface pushes against the sample and is deflected sideways (towards the “pointy side” of the electrode—the side of the tip surface where the point of the tip is located—e.g. in the lower window in section a of FIG. 15 the pointy side would be the lower side of the tip surface). For example, by arranging the plurality of micro-wires in relation to the main needle body such that the surface of the micro-wire and the needle tip surface are parallel, a smoother insertion and maximised area for the sensing site can be achieved.

The method of FIG. 11 may include an additional recess forming step 36 between steps 20 and 22. In step 36, part of the tips of the electrode is dissolved using a solvent to form a recess 40 in the end surface of the electrode 4 as shown in part a) of FIG. 16. For example, the recess can be formed by an electrochemical leaching step (e.g. by dissolving into an electrolyte in the presence of electrical current). The parts of the electrode 4 which are not to be dissolved may be masked by covering them with a mask material, so that only the portion at the end of the tip is dissolved. The subsequent steps of FIG. 11 are then performed on the wire electrodes having the recess in their tips. Therefore, as shown in part b) of FIG. 16, when the impedance reducing layer is subsequently deposited at step 28 of FIG. 11, the nano-structures 42 are deposited on the inside of the recess 40. The nano-structures 42 may also extend onto the surface of the electrode tip outside the recess. When the functionalization layer (e.g. IrOx) is then deposited on top of the impedance reducing layer at step 34, the functionalization layer 44 is deposited inside the recess. The functionalization material may also protrude out of the recess beyond the end of the electrode tip as shown in part C of FIG. 16.

The approach shown in FIG. 16 provides several advantages. Firstly, providing a recess means that a greater volume of iridium oxide or other functionalization material can be deposited at the end of the electrode, which can improve the electrochemical properties of the probe. For example, given the available space, the charge capacity of the iridium oxide layer can be improved up to a 1000 times. Also, this approach provides robustness against mechanical deterioration of the electrode tips. During the working life of the probe, the electrodes may repeatedly be inserted into a sample and removed, and so the tips of the electrodes may gradually be worn away by contact against the sample, which can cause deterioration of the signals measured by the probe. By including the recess and depositing the surface layers inside the recess, then even if the end of the probe is worn down (e.g. so that the surface now is at the position indicated by the line 46 in FIG. 16), then there will still be a layer of the impedance reducing nano-structures and a layer of the functionalization material at the end of the electrodes, so that the electrode can still perform its function. Therefore, the recessed design helps to increase the probe lifetime.

A similar recess may be formed at the back end of the probe, with the connection layer of metal nanostructures formed at least partly inside the recess. Again, this helps provide robustness against mechanical deterioration, which could otherwise wear away the connection layer if the connection layer is repeatedly pressed against the contact bumps of pixel readout circuits as discussed below in relation to FIGS. 18 and 19.

It is also possible to provide the needle with the ability to actively control the direction and/or location of the wire electrodes as the needle is inserted. For example as shown in FIG. 17, the wire electrodes may be disposed within a harness layer 36, along at least part of the length of the wire electrodes (it is not necessary to provide the harness layer 36 along the full length of the electrodes). The insulating material surrounding the electrodes is not shown in FIG. 17 for conciseness—this is still provided. While FIG. 17 shows an example where the wire electrodes are embedded in a continuous matrix of the harness layer 36, it is also possible to provide the harness layer 36 as a membrane or disk which extends around the outside of the wire bundle extends between the respective wires inside the bundle. For example, the disk may be placed around 30-40% of the length of the wire away from the front end of the bundle.

A number of threads 38 (e.g. made of textile) may be attached to the harness layer 36 at different points about the perimeter of the wire bundle. For example, at least three threads may be provided. Each of the threads is attached to a drive unit 39 which controls, separately for each thread, the length of thread between the harness layer 36 and the drive 39. Hence, the drive unit 39 can selectively apply a force to any given thread 38 to pull on the harness layer, thus applying bending of the bundle tip orientation. Hence, depending on which threads the force is applied to, the wire bundle can be “steered” in the desired direction to control the passage of the probe into the sample and cause the wire electrodes to reach the desired location in the sample. In another example, the threads 38 may be replaced with a rod such that structural forces may be applied through such rods to both pull and push the harness layer 36. In this manner, the drive unit 39 may be used to control the rods 38 connected to the harness layer 36 in order to move the locations of the tips of the wire electrodes. The tip of wire electrodes may be placed in a retracted position or an extended position depending on the location of the needle, for example.

FIG. 18 shows an example of an integrated circuit (IC) 50 which can be used to read out and amplify signals measured using the electrochemical probe. The IC 50 may be a multi electrode array (MEA), a CMOS based potentiostat or a pixelated photodetector. All these are already available commercially and therefore it is not necessary to design a bespoke circuit for this purpose, which reduces the cost of implementing an apparatus for electrochemical measurements. As shown in FIG. 18, the IC 50 includes a number of pixel read out circuits 52 arranged in a square or rectangular grid pattern, with each pixel readout circuit including a contact region 54 made from a conductive material (e.g. platinum, gold, indium) connected to an amplifier read out circuit 56. The amplifier circuit may be formed according to any known semiconductor (e.g. CMOS-based) circuit design. The signals amplified by each pixel readout circuit may then be output to a processor, memory or external apparatus for storage or analysis.

FIG. 19 shows how the electrochemical probe 2 may be interfaced with the integrated circuit 50. As shown in FIG. 19, the gold contact bumps 58 at the back ends of the respective wire electrodes 4 can simply be pressed directly against the contact bumps 54 of the respective pixel readout circuits of the IC 50 to provide the electrical connection between the wires and corresponding pixels (without any interposing connector unit between the wire bundle and the IC 50). Hence, the integrated circuit provides a multi-channel amplification and readout system for reading the electrode signals from the respective wires. It is not essential that every pixel readout circuit of the IC 50 interfaces with a corresponding wire electrode. Depending on the arrangement of the wires within the bundle, it is possible that some pixel readout circuits may not contact a corresponding wire.

An alternative is that the wires can also be embedded in a second cladding holding wires together and have the bumps for connection.

As an alternative technique for interfacing the micro-electrodes with read out electronics or a data processing apparatus, the wire bundle may be packaged into an enclosure, but the enclosure does not include an integrated circuit as discussed above for amplifying the signals from the probe. Instead, each wire may be individually bonded or soldered to a corresponding channel of a connector (e.g. a socket or plug). When the needle is in use, the connector can be coupled to an external amplifier or other electronic device for processing the outputs of the electrodes. Hence, it is not necessary for the needle, or at least the needle tip, itself to include circuitry for amplifying or processing the signals read by each electrode.

For wire bundles with relatively low channel count (e.g. less than 1000 wires in the bundle), either the approaches discussed above can be used. However, when the channel count is higher (e.g. greater than 1000 wires), then it becomes increasingly impractical to individually bond each wire to the connector, and in this case the approach shown in FIG. 19 may be more useful, whereby the bumps on the end of each wires are simply pressed against the contact portions of a pixelated integrated circuit.

FIG. 20 is graph showing scaling of stray capacitance of the electrodes with length and inner/outer diameter ratios. FIG. 20 plots stray capacitance against length, for examples with inner/outer diameters (in μm) of 10.7/26.6, 7.1/30.1, 5.4/27.4 and 1.1/28.3 respectively as indicated. The inner diameter refers to the diameter of the conducting core 6 while the outer diameter refers to the total diameter of the wire electrode 4 including the core 6 and the insulating sheath 8. FIG. 21 shows images showing examples with different inner/outer diameter ratios. As shown in FIG. 20, the stray capacitance increases with increasing length of the electrodes, and also increases as the core diameter becomes thicker relative to the outer diameter. Nevertheless, the stray capacitance remains relatively low even for electrodes with relatively long wire lengths (e.g. 3-5 cm). Given the modification of the microwire tips, the coupling capacitance is significantly larger (up to 10×) through the tips; thus the combination of a microwire with 3:1 outer:inner diameter (insulator:core) ratio with the modification protocol surprisingly enables use of several centimetre long wires, which would not be possible with unmodified wires and conventional probes. By allowing longer wires to be produced with less noise due to stray capacitance, this enables the probes to penetrate deeper into the brain or other tissue.

FIG. 22 shows an example in which the wire electrodes 4 (including both their core 6 and the insulating sheathes 8) are partially embedded in a block of cladding material 150 along part of their length, with gaps 152 between the respective wire electrodes along a remaining part of their length. The layer of nanostructures or other modifications at the tips of the electrodes are not shown in FIG. 22 for conciseness, but it will be appreciated that the tips of the electrodes can be modified in the same way as in any of the examples described above. The cladding material 150 provides a rigid support for the probe to prevent separation of the bundle of wire electrodes 4, while providing gaps between the free ends of the electrodes 4 can be useful for allowing some separation of the wire electrodes when inserted into a sample (e.g. the insulating material may be a flexible material), increasing the area over which the wire electrodes can gather measurements or provide stimulation. Although FIG. 22 shows an example where the cladding material 150 is formed at the back end of the probe, in other examples the cladding material 150 could be at a mid-point of the electrodes so that both ends of the wire electrodes may be free to move as there are gaps between the insulating sheathes of the wires.

FIG. 22 shows an example process for manufacturing the bundle of flexible wire electrodes 4 held together by a block of rigid cladding material 150. As shown in the left hand part of FIG. 22, initially the wire electrodes may be formed individually with the core 6 surrounded by a first sheath 8 of insulating material and a concentric second sheath 156 of cladding material outside the first sheath 8. The cladding material may be more soluble to a given solvent than the insulating material used for the first sheath 8. Other than the solubility, it is preferable if the cladding material is similar in physical properties to the insulating material, e.g. similar in thermal expansion and composition. The wires 4 are bundled together parallel to each other, and heat is applied to melt the second cladding layer 156 together to form a block of melted-together cladding 150 with the wires 4 of core material 6 and insulating material 8 embedded inside the cladding block. A solvent which can dissolve the cladding but not the insulation is then applied to dissolve part of the cladding block 150 down to a given level, preserving the initial ordering and parallel stacking of the wires and leaving the free end of the wires with gaps 152 in between while a bound section of wires is surrounded by the cladding block 150. These steps are valid in a range of possible geometries, e.g. hemispherical, saw-like, planar, random or combined.

In addition to the wire electrodes 4 having a conducting core 6 surrounded by an insulating sheath 8, the needle 2 may also include hollow-core channels 170 which comprise a hollow core (made of air) surrounded by a sheath of the same insulating material 8 as used for the wire electrodes 4. The hollow-core channels 170 are arranged parallel to the wire electrodes 4. For example, the hollow-core channels 170 can be formed by providing some wires in the bundle with a solid core of soluble material clad in an insulating sheath, and bundling these together with the conducting-core electrodes 4 in a desired pattern, and then dissolving the core of the hollow-core channels 170 to leave an empty void at the center of these channels. The hollow-core fibers 170 can be useful for both delivery and extraction of liquid or almost liquid phase substances from the surface or interior of virtually any biological media. For example, they could be used as micro-fluidic channels for local delivery of pharmaceuticals, molecules, cells, genes, tissue, etc. By combining the hollow-core fibers with the conducting-core UMEs in the same probe, this can be used for cellular sampling for local ablation or post-hoc examination purposes, e.g. of neurons from which electrophysiological recording has been achieved. Hence, without needing to remove the electrochemical needle and inserting a separate liquid delivery/sampling needle, a single needle can be used for both purposes. The hollow channels can be used for both liquid delivery and liquid extraction. An array of hollow channels combining both modalities in parallel (delivery and extraction, e.g. with half of the hollow-core channels used for delivery and the other half for suction) can form the basis of e.g. biopsies and ablation. Given their micron-size features, arrays of hollow fibers could also represent implantable scaffolds for promoting cell growth.

In some cases, an array of micro-fibres which all comprise hollow cores could be provided, without any of the fibres having conducting cores. In this case, the use of the Taylor-Ulitovsky process for manufacturing the sheathes of the hollow-core channels can still be useful for reducing the tissue damage when the probe is inserted into the tissue.

In summary, by providing a multimodal needle comprising a plurality of micro-electrodes for electrochemical sensing, each micro-electrode comprising a core of conducting material, insulating material surrounding the core, and a layer of metal or metal oxide nano-structures deposited on tips of the micro-electrodes at a first end for interfacing with a target sensing site, a localized sensing needle having a high signal to noise ratio of electrochemical signals due to lower impedance at the first end. Thus, the multimodal needle is able to provide a high resolution localized monitoring in the regions surrounding different portions of the needle.

One proposed use for the multimodal needle can be where the needle is used to sense characteristics of the tissue in which the needle is inserted, so that it can be verified that the location of the needle is the correct location at which treatment is needed. Then, treatment can be applied using the needle, e.g. by applying a stimulation current to heat the tissue at the target site, or by delivering fluid medicament through the needle. After treatment, further readings measured using the multimodal needle could then be used to detect the effectiveness of the treatment.

For example, the needle could be used in the field of oncology for detection and treatment of cancerous tumours. Tumours may be distinguished from healthy tissue by their oxygen content, which may result in the tumours having a different pH from the healthy tissue. Malign and benignant tumours may also be distinguished by their pH. A multimodal needle with a bundle of micro-electrodes as discussed above, with the tips of the electrodes deposited with a first layer of gold nano-structures and a functionalisation layer of iridium oxide (IrOx), can be used to detect pH in the tissue in which the needle is inserted. FIGS. 23 and 24 show experimental results to demonstrate the effectiveness of pH detection using such micro-electrodes.

FIG. 23 shows pH/mV sensor response plot for a probe based on these micro-electrodes when dipped in commercial pH calibration solutions at room temperature. These measurements are based on open circuit voltage measurements of the IrOₓ response when the probe is simply inserted into the solution (no stimulation current or voltage is applied). Each box contains the average of response to 10 consecutive cycles for stability. Approximately 70 mV/pH Nerstian response has been recorded. As shown in FIG. 23, clear differences in the voltage measured can be seen for the respective pH levels. FIG. 24 shows cyclic voltammetry measurements for a probe comprising the micro-electrodes discussed above, when the voltage is swept between −500 mV and 600 mV vs a reference electrode in different commercial pH calibration solutions at 50 mVs⁻¹ sweeping rate in a standard three-electrode cell. Again, different response curves are seen for the respective pH values, showing that it is feasible to detect pH with the multimodal needle.

Hence, a surgeon can insert the needle into tissue, and use the pH measurements to verify whether the needle is inserted into healthy or cancerous tissue before proceeding with subsequent treatment. If the surgeon has verified that the needle is inserted at the correct tumour location, the surgeon can then apply treatment by supplying a stimulation current to the micro-electrodes of the multimodal needle, to heat the cancerous tissue to cause ablation. After performing the ablation, the effectiveness of the ablation can then be verified by further measurements using the multimodal needle.

FIG. 25 shows an image of an in-vitro experiment using a pork liver, for validating that it is feasible for the needle comprising micro-electrodes as discussed above to detect altered tissue during simulated thermal ablation. As shown in the upper part of FIG. 25, ablated regions were formed in the pork liver using a standard commercially available heating element, using a controlled temperature of 55° C. for 6 minutes at one site, and also using an uncontrolled ablation at 110° C. at another site. The ablated regions are the circular discoloured regions surrounding the large circular holes at the upper part of the image, where the hole was caused by penetration of the heating element into the tissue. The other holes in the pork liver are locations of blood vessels or locations at which the needle has been inserted for sensing measurements. Measurements were taken in both the unaffected and discoloured (unablated and ablated) regions. In the centre of the image of FIG. 25, the probe is seen during recording in a healthy unablated region.

A sine wave stimulation was applied to the sample in the healthy and ablated regions, at a range of different frequencies, and the impedance measured using the probe in each case. FIG. 26 shows the sensor response in the unablated healthy tissue (upper line of the plot), tissue ablated to normal clinical levels (middle line) and damaged tissue at uncontrolled ablation at 110° C. (lower line). FIG. 26 shows that there is a clear difference in impedance measured in the healthy and ablated tissue, across the entire range of frequencies from 20 kHz to 90 kHz. To demonstrate reproducibility of the results, FIG. 27 shows results of further measurements of healthy and ablated tissue when stimulated using a sine wave of frequency 10 kHz. As shown in FIG. 27, the repeated measurements cluster at distinct levels for the healthy and ablated tissue, showing that a reliable detection of whether the tissue is ablated or unablated is possible using the probe.

Hence, these results show that is feasible to use a single multimodal needle to (a) detect whether the site in which the needle is inserted is the correct location, (b) deliver treatment to that site, and (c) make further measurements to check the effectiveness of the treatment.

In the present application, the words “configured to . . . ” are used to mean that an element of an apparatus has a configuration able to carry out the defined operation. “Configured to” does not imply that the apparatus element needs to be changed in any way in order to provide the defined operation.

Although illustrative embodiments of the invention have been described in detail herein with reference to the accompanying drawings, it is to be understood that the invention is not limited to those precise embodiments, and that various changes and modifications can be effected therein by one skilled in the art without departing from the scope and spirit of the invention as defined by the appended claims.

1. A multimodal needle comprising a plurality of micro-electrodes for electrochemical sensing, wherein each micro-electrode comprises:
a core of conducting material,
insulating material surrounding the core, and
a layer of metal or metal oxide nano-structures deposited on a tip of a first end of the micro-electrode for interfacing with a target sensing site.

2. A multimodal needle according to claim 1, wherein the multimodal needle comprises an opening at a tip of a first end of the needle and a through passage for delivering or removing fluid.

3. A multimodal needle according to claim 1, comprising a functionalization layer deposited on the layer of metal or metal oxide nano-structures at said first ends of the micro-electrodes.

4-5. (canceled)

6. A multimodal needle according to claim 3, comprising a first micro-electrode with a first type of functionalization layer deposited on the layer of metal or metal oxide nano-structures at said first end, and a second micro-electrode with a second type of functionalization layer deposited on the layer of metal or metal oxide nano-structures at said first end.

7. A multimodal needle according to claim 3, wherein the functionalization layer comprises self-assembled monolayers.

8. A multimodal needle according to claim 1, wherein the plurality of micro-electrodes are disposed in a common insulating matrix of the insulating material.

9. A multimodal needle according to claim 1, wherein the micro-electrodes have a diameter less than or equal to 25 μm.

10. A multimodal needle according to claim 1, wherein a first end of the needle has a sharpened tapered end, having a first end needle tip surface.

11. A multimodal needle according to claim 1, wherein the first end micro-electrode tips have a polished angled tip surface with a sharpened point, wherein the metal or metal oxide nano-structures are formed on the first end micro-electrode tip surface or in the recess of the first end micro-electrode tip surface.

12. A multimodal needle according to claim 10, wherein the first end needle tip surface is in plane with the first end micro-electrode tip surface.

13. A multimodal needle according to claim 1, wherein the first end tips of the micro-electrodes are movable in relation to a needle tip.

14. A multimodal needle according to claim 13, wherein the first end tips of the micro-electrodes are movable between a retracted position, in which the first end tips of the micro-electrodes are retracted within the needle through passage, and an extended position, in which the first end tips of the micro-electrodes are extended further than the first end needle tip.

15. A multimodal needle according to claim 1, wherein any one or more of:
at least one of the micro-electrodes are provided within the needle through passage;
at least one of the micro-electrodes are provided on the outer surface of a main needle tube body surrounding the needle through passage;
at least one of the first end tips of the micro-electrodes are provided around an internal periphery of the first end needle tip; and/or
at least one of the first end tips of the micro-electrodes are provided around an external periphery of the first end needle tip.

16-18. (canceled)

19. A multimodal needle according to claim 1, wherein the first end tips of the micro-electrodes are provided at different positions on the outer surface along the length of the needle tip and needle body.

20. A multimodal needle according to claim 1, comprising hollow-core channels arranged in parallel with the micro-electrodes.

21. (canceled)

22. A multimodal needle according to claim 1, wherein the multimodal needle is epidural needle.

23. A multimodal needle according to claim 1, wherein the multimodal needle comprises a portion for coupling with a radio frequency generator.

24. A plurality of electrochemical sensor micro-electrodes, each micro-electrode comprising:
a core of conducting material,
insulating material surrounding the core, and
a layer of metal or metal oxide nano-structures deposited on tips of the micro-electrodes at a first end for interfacing with a target sensing site;
wherein the micro-electrodes are disposed in a common insulating matrix of the insulating material, and the micro-electrodes are connectable to a hypodermic needle to form a multimodal needle according to claim 1.

25. A system comprising a multimodal needle according to claim 1 and an integrated circuit comprising a plurality of contact portions to receive an electrode signal, and an amplifying portion to amplify the electrode signal received at the contact portions, wherein connection layers of metal nano-structures on the tips of the micro-electrodes at the second end are in contact with corresponding contact portions of the integrated circuit.

26. A system according to claim 25, comprising a radio frequency generator for coupling with the multimodal needle.
